(19) United States
(12) Patent Application Publication
(10) Pub. No.: US 2025/0206106 A1
(43) Pub. Date: Jun. 26, 2025

(54) VEHICLE FRAGRANCE DEVICE AND VEHICLE

(71) Applicant: Ford Global Technologies, LLC, Dearborn, MI (US)

(72) Inventors: Shuhan Ma, Nanjing (CN); Wei Xu, Nanjing (CN); Zhenhai Kong, Nanjing (CN); Xu Zhengdong, Nanjing (CN)

(73) Assignee: Ford Global Technologies, LLC, Dearborn, MI (US)

(21) Appl. No.: 18/942,872

(22) Filed: Nov. 11, 2024

(30) Foreign Application Priority Data
Dec. 21, 2023 (CN) 202311779586.1

(51) Int. Cl. B60H 3/00; A61L 9/12

(52) U.S. Cl. CPC B60H 3/0007; A61L 9/125; A61L 2209/134

(57) ABSTRACT

A vehicle fragrance device has a fragrance assembly comprising a fragrance unit with a fragrance medium, a carrier for carrying the fragrance unit and a sealing unit. An actuating assembly comprises a movable actuator that triggers the sealing unit to switch the fragrance assembly between a sealed state and a non-sealed state. After triggering the sealing unit, the movable actuator disengages from the sealing unit and the fragrance assembly remains in the triggered state.

### CROSS-REFERENCE TO RELATED APPLICATION

This application claims foreign priority benefits under 35 U.S.C. § 119 (a)-(d) to China Application No. 202311779586.1 filed Dec. 21, 2023, which is hereby incorporated by reference in its entirety.

### FIELD OF THE DISCLOSURE

The present disclosure generally relates to motor vehicles, and more specifically relates to a fragrance unit and a vehicle fragrance device for a motor vehicle.

### BACKGROUND OF THE DISCLOSURE

Fragrance distribution devices have been proposed for use in a motor vehicle. For example, a fragrance distribution device may be configured to distribute fragrance from a fragrance medium to air in the vehicle. The fragrance distribution device generally may include an actuator having a mechanism configured to actuate a valve device for each fragrance containing box between a sealed state for blocking passage of air through a fragrance medium of the fragrance containing box and an unsealed state for allowing air to pass through the passage of the fragrance medium of the fragrance containing box towards an outlet. It would be desirable to provide for enhanced fragrance distribution in a vehicle.

### SUMMARY OF THE DISCLOSURE

According to a first aspect of the present disclosure, a vehicle fragrance device has a fragrance assembly comprising a fragrance unit with a fragrance medium, a carrier for carrying the fragrance unit and a sealing unit. An actuating assembly comprises a movable actuator that triggers the sealing unit to switch the fragrance assembly between a sealed state and a non-sealed state. After triggering the sealing unit, the movable actuator disengages from the sealing unit and the fragrance assembly remains in the triggered state.

Embodiments of the first aspect of the present disclosure can include any one or a combination of the following features:
the sealing unit further comprises a button and a push lock connected to the button, wherein the button has a first position corresponding to the sealed state and a second position corresponding to the non-sealed state, and wherein the button is configured to trigger the actuator to switch the button between the first position and the second position, and the push lock locks the button in the triggered position;
the carrier comprises a bottom wall and a peripheral wall around the bottom wall, wherein the carrier further comprises a peripheral wall extension end, the peripheral wall extension end extends away from the fragrance unit from the bottom wall and has an inner inclined surface adapted to the button and facing the button;
the carrier has a first accommodating chamber for accommodating the fragrance unit and a second accommodating chamber located at an end of the carrier and being in communication with the first accommodating chamber, and the sealing unit is located in the second accommodating chamber;
the carrier has a first opening facing the movable actuator, and the sealing unit seals the first opening in the sealed state;
the sealing unit further comprises a button and an elastic seal located between the opening and the button, and the elastic seal is arranged around at least one of an outer peripheral wall of the button and an inner peripheral wall of the opening;
the elastic seal comprises a sealing portion located at an end and a guide groove adjacent to the sealing portion, and the guide groove is located on one side of the elastic seal facing a movement direction of the elastic seal;
the sealing unit further comprises a receiving groove arranged on the outer peripheral wall of the button or the inner peripheral wall of the opening, wherein the sealing portion rests against the receiving groove, the guide groove and the receiving groove are located on the same side of the elastic seal, and the sealing portion is partially accommodated in the receiving groove in the sealed state;
the carrier has an air inlet and an aroma outlet, and the sealing unit is used to seal at least one of the air inlet and the aroma outlet;
an airflow channel for airflow to pass through and a control valve provided in the airflow channel;
the actuating assembly further comprises an actuating rod, on which the movable actuator is arranged, and the movable actuator is configured to perform reciprocating motion along an extension direction of the actuating rod to trigger or disengage from the sealing unit;
wherein the actuating assembly comprises multiple linearly and uniformly arranged actuators, and a spacing between the actuators is not equal to a spacing between the fragrance assemblies;
the spacing between the actuators is smaller than the spacing between the fragrance assemblies;
multiple actuators are nonlinearly arranged on the actuating rod;
multiple fragrance assemblies are arranged nonlinearly, and the actuator is configured such that when one of the fragrance assemblies is in the non-sealed state, the remaining fragrance assemblies are in the sealed state;
the actuating assembly further comprises an actuating rod, on which the actuator is mounted, and the movable actuator is configured to perform rotational motion along a central axis of the actuating rod to trigger or disengage from the sealing unit;
multiple actuators adapted to the positions of the fragrance assemblies are provided, and projections of the multiple actuators on a plane perpendicular to the central axis of the actuating rod are at least partially offset;
the fragrance assembly further comprises a cover comprising a main body portion and a connecting portion that is connected to the carrier on one side in a sealed manner and to the fragrance unit on another side; and
the fragrance unit comprises a fragrance box, wherein the fragrance medium is located inside the fragrance box, and a first fragrance retention gap is formed between the fragrance box and the carrier.

According to a second aspect of the present disclosure, a vehicle has a fragrance device located inside the vehicle comprising a housing that at least partially accommodates a fragrance assembly. The fragrance assembly has a fragrance unit with a fragrance medium, a carrier for carrying the fragrance unit, and a sealing unit and an actuating assembly comprising a movable actuator. The movable actuator triggers the sealing unit to switch the fragrance assembly between a sealed state and a non-sealed state. After triggering the sealing unit, the movable actuator disengages from the sealing unit and the fragrance assembly remains in the triggered state.

These and other features, advantages, and objects of the present disclosure will be further understood and appreciated by those skilled in the art by reference to the following specification, claims, and appended drawings.

### BRIEF DESCRIPTION OF THE DRAWINGS

For a better understanding of the present disclosure, reference may be made to embodiments shown in the following drawings. The components in the drawings are not necessarily to scale and related elements may be omitted, or in some instances proportions may have been exaggerated, so as to emphasize and clearly illustrate the novel features described herein. In addition, system components can be variously arranged, as known in the art. Further in the figures, like reference numbers refer to like parts throughout the different figures.

FIG. 1 is a perspective view of a vehicle having a vehicle fragrance device located inside the vehicle according to an embodiment of the present disclosure;

FIG. 2 is a partial upper perspective view of the fragrance device according to an embodiment of the present disclosure;

FIG. 3 is a partial cross-sectional view taken centrally through a fragrance assembly of the fragrance device according to an embodiment of the present disclosure;

FIG. 4 is a side view of the vehicle fragrance device showing one of fragrance assemblies being triggered according to an embodiment of the present disclosure;

FIG. 5 is a side view of the vehicle fragrance device showing another fragrance assembly being triggered according to an embodiment of the present disclosure;

FIG. 6 is a partial lower perspective view of an actuating assembly driving the fragrance assembly according to an embodiment of the present disclosure;

FIG. 7 is a partial schematic diagram of the actuating assembly driving the fragrance assembly according to another embodiment of the present disclosure;

FIG. 8 is a partial side schematic diagram of the fragrance device according to another embodiment of the present disclosure;

FIG. 9 is a partial cross-sectional view of the fragrance assembly in a sealed state according to an embodiment of the present disclosure;

FIG. 10 is an enlarged view of Section A shown in FIG. 9 at point A;

FIG. 11 is a partial cross-sectional view of the fragrance assembly in a non-sealed state according to an embodiment of the present disclosure;

FIG. 12 is an enlarged view of Section B shown in FIG. 11;

FIG. 13 is a partial cross-sectional view of the fragrance assembly in the non-sealed state according to another embodiment of the present disclosure;

FIG. 14 is an enlarged view of Section C shown in FIG. 13;

FIG. 15 is a cross-sectional view of the fragrance assembly showing an airflow path in the non-sealed state according to an embodiment of the present disclosure; and

FIG. 16 is an enlarged view of section D shown in FIG. 15.

### DETAILED DESCRIPTION OF PREFERRED EMBODIMENTS

Embodiments of the present disclosure are described below. However, it should be understood that the disclosed embodiments are only examples, and other embodiments may take various and alternative forms. Drawings are not necessarily drawn to scale; and some functions may be exaggerated or minimized to show details of specific components. Therefore, the specific structural and functional details disclosed herein should not be interpreted as restrictive, but merely as a representative basis for teaching those skilled in the art to use the present disclosure in various ways. As will be understood by those of ordinary skill in the art, various features shown and described with reference to any one of the drawings may be combined with features shown in one or more other drawings to produce embodiments which is not explicitly shown or described. The combination of the features shown provides a representative embodiment for a typical application. However, various combinations and modifications of features consistent with the teachings of the present disclosure may be expected for certain specific applications or embodiments.

In this application document, when an element or part is referred to as being “on . . . ”, “bonded to”, “connected to”, or “coupled to” another element or part, the element or part can be directly on the another element or part, can be bonded, connected or coupled to the another element or part, or there may be intervening elements or parts. In contrast, when an element is referred to as being “directly on . . . ”, “directly bonded to”, “directly connected to”, or “directly coupled to” the another element or part, the intervening elements or parts may not be present. Other words used to describe the relationship between elements should be interpreted in a like fashion.

The inventors of the present disclosure realized that vehicle fragrance devices in the prior art have shortcomings in terms of fragrance leakage and uniformity of fragrance release. Based on the problems in the prior art, the inventors of the present disclosure proposes an improved vehicle fragrance device and a vehicle, in order to solve the problems in the prior art.

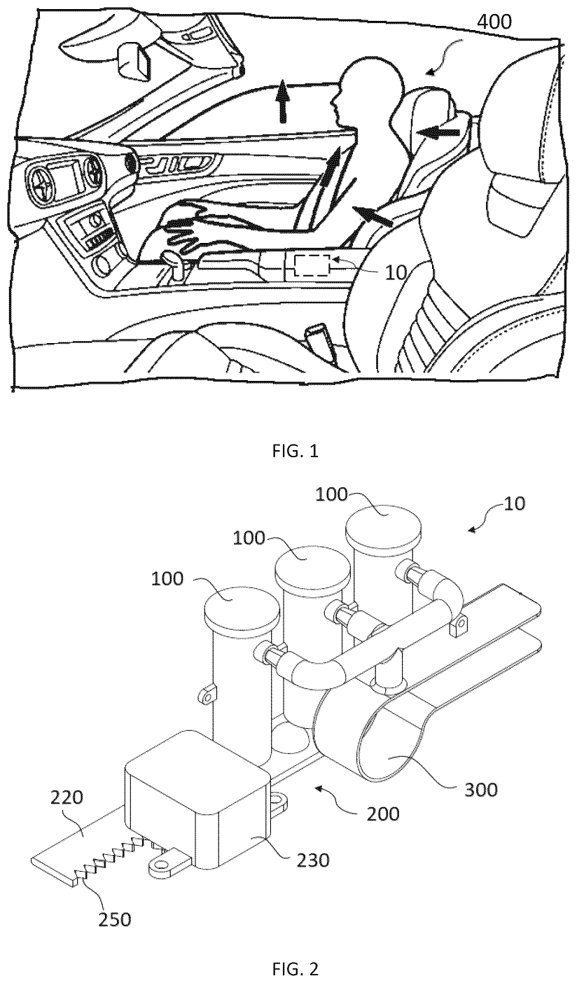

FIG. 1 generally illustrates a partial schematic diagram of a vehicle 400 with a vehicle fragrance device 10 according to one or more embodiments of the present disclosure. It should be understood that in the context of the present disclosure, the vehicle implementing the present disclosure may refer to any means of transportation, including but not limited to fossil fuel vehicles, electric vehicles (such as plug-in hybrid electric vehicles (PHEVs), full hybrid electric vehicles (FHEVs), mild hybrid electric vehicles (MHEVs), or battery electric vehicles (BEVs)), and so on. The vehicles may include mobility related components such as engine, electric motor, transmission, suspension, driveshaft, and/or wheels. The vehicles can be non-autonomous, semi-autonomous (for example, some conventional motion functions are autonomously controlled by the vehicle), or autonomous (for example, motion functions are autonomously controlled by the vehicle without direct user input).

Referring to FIGS. 2 to 8, a vehicle fragrance device 10 is illustrated according to one aspect of the present disclosure. The vehicle fragrance device 10 includes a housing that at least partially accommodates a fragrance assembly 100, the fragrance assembly 100 comprising a fragrance unit 110 with a fragrance medium 112, a carrier 120 for carrying the fragrance unit 110, and a sealing unit 130, and an actuating assembly 200 comprising a movable actuator 210. The actuator 210 triggers the sealing unit 130 to switch the fragrance assembly 100 between a sealed state and a non-sealed state. After triggering the sealing unit 130, the actuator 210 disengages from the sealing unit 130 and the fragrance assembly 100 remains in the triggered state.

The fragrance assembly 100 thus has the sealed state and the non-sealed state under the action of the sealing unit 130. In the sealed state, the sealing unit 130 seals the fragrance assembly 100, obstructing airflow from flowing out of the fragrance assembly 100 after passing through the fragrance medium 112. In the non-scaled state, the sealing unit 130 releases the sealing on the fragrance assembly 100, and fragrance flowing through the fragrance medium 112 can flow out into an interior of the vehicle 400.

The actuator 210 has corresponding triggered and non-triggered positions, and can move between the triggered position and the non-triggered position. When the actuator 210 is moved to the triggered position, the actuator 210 triggers the corresponding sealing unit 130 to enable the fragrance assembly 100 to switch between the sealed state and the non-sealed state. When in the non-triggered position, the actuator 210 does not trigger the sealing unit 130 and the state of the fragrance assembly 100 does not switch.

After the actuator 210 triggers the sealing unit 130, it moves away and disengages from the sealing unit 130, and the fragrance assembly 100 remains in the triggered state. In one embodiment, when the fragrance assembly 100 is in the non-scaled state, the actuator 210 moves to the triggered position to trigger the sealing unit 130, and the fragrance assembly 100 switches to the sealed state. After the actuator 210 triggers the scaling unit 130, it moves away and disengages from the sealing unit 130. The actuator 210 does not need to constantly contact the scaling unit 130, nor does the actuator 210 need to remain in the triggered position. At this time, the fragrance assembly 100 is still in the sealed state, obstructing the airflow from flowing out of the fragrance assembly 100 after passing through the fragrance medium 112. When a user needs to use fragrance, the actuator 210 is moved to the triggered position and triggers the sealing unit 130 again. After the actuator 210 triggers the sealing unit 130, the actuator 210 moves away and disengages from the sealing unit 130. The fragrance assembly 100 switches to the non-sealed state and remains in the non-sealed state. The fragrance flowing through the fragrance medium 112 can then flow into the interior of the vehicle 400.

The actuating assembly 200 may also include an actuating rod 220 and a motor 230 for driving the rod to move. The actuator 210 is mounted on the actuating rod 220 and moves with the actuating rod 220. The actuator 210 also includes a gear 240 connected to the motor 210 and a rack 250. The motor 230 can drive the gear 240 to rotate, and then drive the rack 250 to move through the gear 240, thereby driving the actuating rod 220 to start moving. The actuating rod 220 can then drive the actuator 210 to move. Through the cooperation between the actuator 210 and the sealing unit 130, the fragrance assembly 100 is turned on or off to manage the fragrance. It should be understood that the motor can include various types of motors, including but not limited to brushless motor, DC motor, AC motor, stepper motor, AC asynchronous motor, etc.

After triggering the sealing unit 130, the actuator 210 is moved away and the fragrance assembly 100 remains in the triggered position. This eliminates the need for the actuator 210 to remain in the triggered position continuously, which can reduce driving energy required for the actuator 210 to remain in the triggered position continuously. In particular, in some embodiments, the actuator 210 is typically driven by the motor 230 and can disengage from the sealing unit 130 after triggered, the motor 230 does not need to continuously drive the actuator 210 to remain it in the triggered position, which can reduce the possibility of noise generated by continuous motor driving and improve user experience.

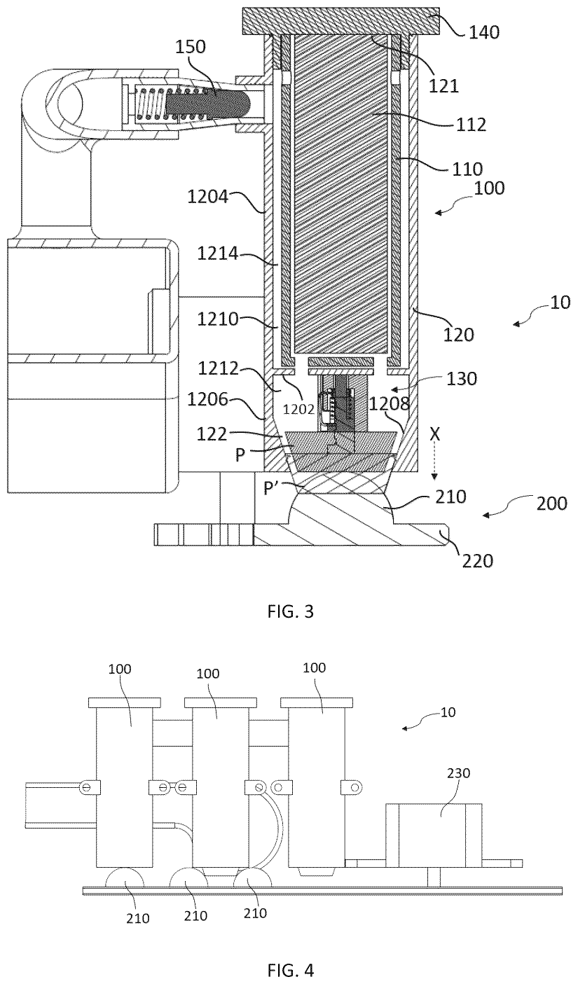

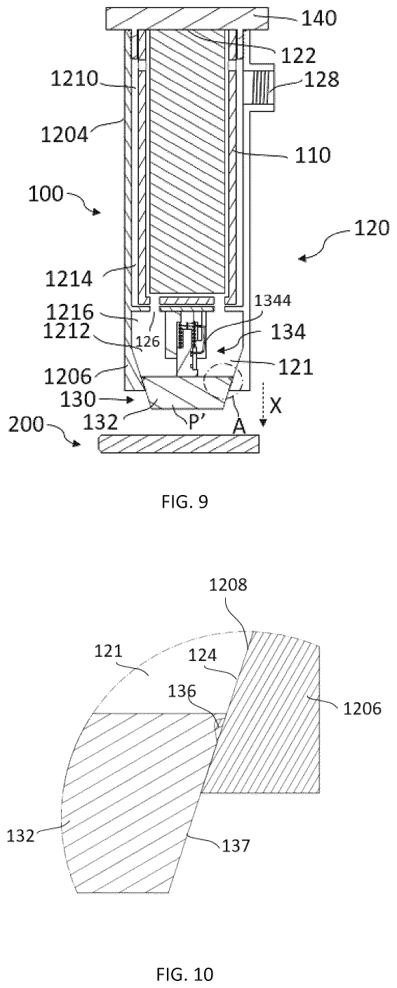

As shown in FIGS. 3 and 9, the carrier 120 may include a first accommodating chamber 1210 for accommodating the fragrance unit 110, an air inlet 126, and an aroma outlet 128, with the fragrance unit 110 located within the first accommodating chamber 1210. The airflow enters the first accommodating chamber 1210 through the air inlet 126, flows through the fragrance medium 112 in the fragrance unit 110, and then flows out into the vehicle through the aroma outlet 128.

The fragrance unit 110 also includes a fragrance box 114, and the fragrance medium 112 is located inside the fragrance box 114. The fragrance box 114 is located inside the first accommodating chamber 1210, and a first fragrance retention gap 1214 is formed between the fragrance box 114 and the carrier 120. The first fragrance retention gap 1214 is in fluid communication with an airflow channel 1218. When the fragrance assembly 100 is in an unused state, no airflow can be formed. The fragrance naturally evaporated from the fragrance medium 112 can be retained in the first fragrance retention gap 1214. When the fragrance assembly 100 is turned on, the fragrance retained in the first fragrance retention gap 1214 can flow out of the aroma outlet 128 into the vehicle in a short period of time, which can enhance the efficiency of aroma emission, reduce response time, and further enhance user experience and satisfaction.

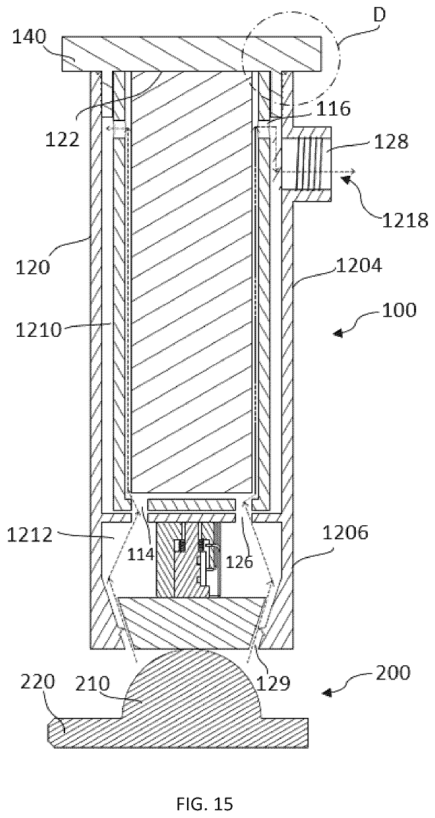

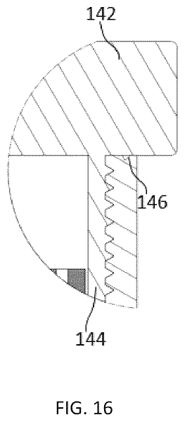

As shown in FIGS. 15 and 16, the fragrance assembly 100 further includes a cover 140 for closing a second opening 122, and the cover 140 is sealingly connected to the carrier 120. Wherein, the cover 140 includes a main body portion 142 and a connecting portion 144 that extends from the main body portion 142 towards the first accommodating chamber 1210. The main body portion 142 can cover the second opening 122, the connecting portion 144 is scalingly connected to the carrier 120 on one side and connected to the fragrance unit 110 on the other side. In one embodiment, a seal 146 is provided below the main body portion 142, and the seal 146 is arranged around the connecting portion 144. This prevents airflow from flowing out through the installation gap between the cover 140 and the carrier 120 when the fragrance assembly 100 is in the sealed state, further reducing the possibility of fragrance leakage in the sealed state.

As shown in FIGS. 3, 9, and 11, the carrier 120 includes a bottom wall 1202 and a peripheral wall 1204 around the bottom wall 1202. The peripheral wall 1204 extends from the bottom wall 1202 towards the cover 140, and the bottom wall 1202 and the peripheral wall 1204 define the first accommodating chamber 1210. The fragrance unit 110 is located within the first accommodating chamber 1210.

The carrier 120 also includes a peripheral wall extension end 1206. The peripheral wall extension end 1206 extends away from the cover 140 from the bottom wall 1202. It can also be understood that the peripheral wall extension end 1206 extends away from the fragrance unit 110 from the bottom wall 1202. The peripheral wall extension end 1206 has an inner inclined surface 1208 that is adapted to the button 132 and faces the button. This allows the peripheral wall extension end 1206 to adapt to the button 132.

As shown in FIGS. 9 to 16, the carrier 120 also includes a second accommodating chamber 1212 adjacent to and in communication with the first accommodating chamber 1210. The peripheral wall extension end 1206 and the bottom wall 1202 define the second accommodating chamber 1212. The sealing unit 130 is located inside the second accommodating chamber 1212. In the unsealed state, the airflow enters the second accommodating chamber 1212, then enters the first accommodating chamber 1210 through the air inlet 126, then flow through the fragrance medium 112 in the fragrance unit 110, and then flow out into the vehicle through the aroma outlet 128.

The carrier 120 has a first opening 121 facing the actuator 210 and a second opening 122 away from the actuator 210. The fragrance unit 110 is placed into or removed from the first accommodating chamber 1210 through the second opening 122.

The first opening 121 is located at an end of the carrier 120 and is opposite to the second opening 122. The first opening 121 is located at one end of the second accommodating chamber 1212 facing the actuator 210. In the sealed state, the scaling unit 130 seals the first opening 121, preventing airflow from entering the second accommodating chamber 1212. The scaling unit 130 seals the second accommodating chamber 1212 to form a second fragrance retention area 1216, where the fragrance naturally evaporated from the fragrance medium 112 can be retained. When the vehicle fragrance assembly 100 is turned on, the fragrance retained in the second fragrance retention area 1216 can flow out into the vehicle in a short period of time, which can enhance the efficiency of aroma emission, reduce response time, and further enhance user experience and satisfaction.

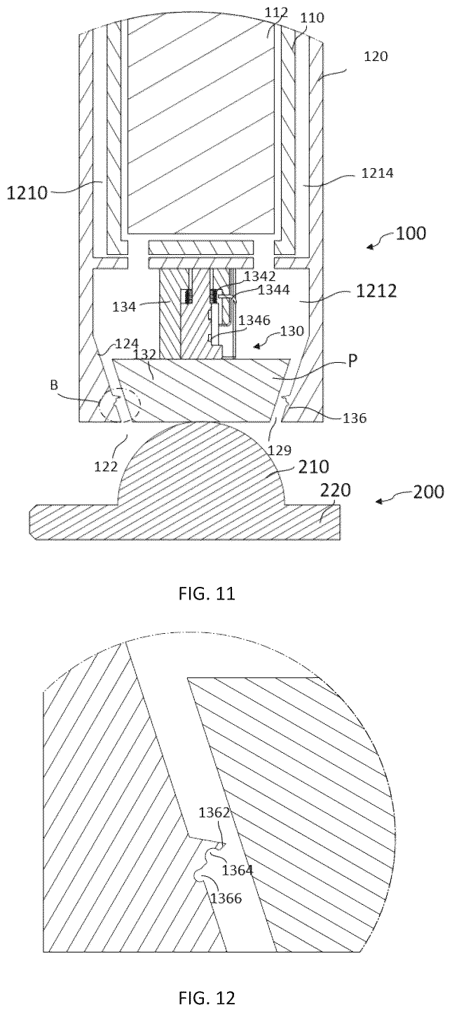

The button 132 has a shape that is adapted to the second opening 122. In one embodiment, as shown in the sectional views of the fragrance assembly 100 along its central axis in FIGS. 9 and 11, the button 132 has an inverted trapezoidal shape, and the second opening 122 has a trapezoidal shape that is adapted to the button 132. Technicians in this field can understand that the button 132 and the second opening 122 also have other suitable shapes.

Placing the sealing unit 130 inside the second accommodating chamber 1212 not only makes the fragrance assembly 100 more compact in structure, but also prevents the scaling unit 130 from being exposed to the outside, which can protect the sealing unit 130. Furthermore, by sealing the first opening with the sealing unit, the sealing of the air inlet 126 can be achieved, which is beneficial for the sealing of the air inlet 126, structurally casier to achieve, and has a better sealing effect, which can further reduce the possibility of fragrance leakage.

The sealing unit 130 is used to seal the air inlet 126 and/or the aroma outlet 128. In one embodiment, the sealing unit 130 is installed at the air inlet 126 or the aroma outlet 128. In the sealed state, the airflow cannot pass through the air inlet 126 or the aroma outlet 128, thus preventing the formation of airflow. In another embodiment, sealing units 130 are respectively installed at the air inlet 126 and the aroma outlet 128. In the sealed state, the air inside and outside the first accommodating chamber 1210 will not exchange, which better reduces the possibility of fragrance leakage and enhances the user experience.

As shown in FIGS. 9 and 11, the sealing unit 130 may also include a button 132 and a push lock 134 connected to the button 132. When the actuator 210 moves to the triggered position, it can trigger the button 132, causing it to move. The push lock 134 can lock and release the button 132, and keep it in the triggered position.

As shown in FIG. 3, the button 132 has a first position P′ corresponding to the sealed state and a second position P corresponding to the non-sealed state. The actuator 210 triggers the button 132 to switch the button 132 between the first position P′ and the second position P, and the push lock 134 can keep the button 132 in the triggered position. In one embodiment, as shown in FIG. 3, the first position P′ may be a released state of the button 132, and the second position P may be a locked state of the button 132. In another embodiment, the first position may be the locked state of the button 132, and the second position may be the released state of the button 132.

It should be understood that the push lock 134 in this application can be implemented in various ways. For example, the push lock 134 can include the push lock mechanism disclosed in U.S. Pat. No. 10,941,590B2, which is hereby incorporated herein by reference, or other push locks currently available on the market that can achieve self-locking function, such as a ESB30 push switch, commercially available from Panasonic.

In one embodiment, as shown in FIGS. 9 and 11, the push lock 134 includes a spring 1342, a sliding rod 1344, and a locking piece 1346 that is connected to the sliding rod 1344 in a movable manner. The sliding rod 1344 cooperates with the locking piece 1346 in a movable manner, and the sliding rod 1344 is held in different positions by the locking piece 1346. As shown in FIG. 9, when the fragrance assembly 100 is in the sealed state, the button 132 is in the first position P′. If the user wants to use fragrance, he needs to switch the fragrance assembly 100 to the non-sealed state. At this time, the actuator 210 is moved to the triggered position to trigger the button 132, the spring 1342 is compressed into an energy storage state, the button 132 is pushed from the first position P′ to the second position P by the actuator 210, the button 132 is held in the second position P through the cooperation of the sliding rod 1344 and the locking piece 1346, and the fragrance assembly 100 is switched into the non-sealed state, as shown in FIG. 11. If the fragrance assembly 100 needs to switch from the non-sealed state to the sealed state, the actuator 210 is moved to the triggered position to trigger the button 132 again, the spring 1342 is released, the actuator 210 is pulled back from the second position P to the first position P′, and the fragrance assembly 100 is switched into the non-sealed state.

As shown in FIG. 3, the button 132 can move between the first position P′ and the second position P along an extension direction of the carrier 120, i.e., a first direction X. As shown in FIGS. 9 and 10, when the fragrance assembly 100 is in the sealed state, the button 132 is in the first position P′, at this time, part of the button 132 passes through the second opening 122 and is exposed outside the second opening 122, and an outer peripheral wall 137 of the button 132 at least partially adheres to an inner peripheral wall 124 of the second opening 122; as shown in FIGS. 11 to 14, when the fragrance assembly 100 is in the non-sealed state, the button 132 is in the second position P, which is far away from the actuator 210 relative to the first position P′, and the airflow can flow into the fragrance assembly 100 through the gap 129 between the outer peripheral wall 137 of the button 132 and the inner peripheral wall of the second opening 122. FIG. 16 shows an airflow path direction in the non-sealed state. The airflow enters the second accommodating chamber 1212 through the gap 129, then enters the first accommodating chamber 1210 through the air inlet 126, flows through the fragrance medium 112, and finally flows out of the cabin through the aroma outlet 128.

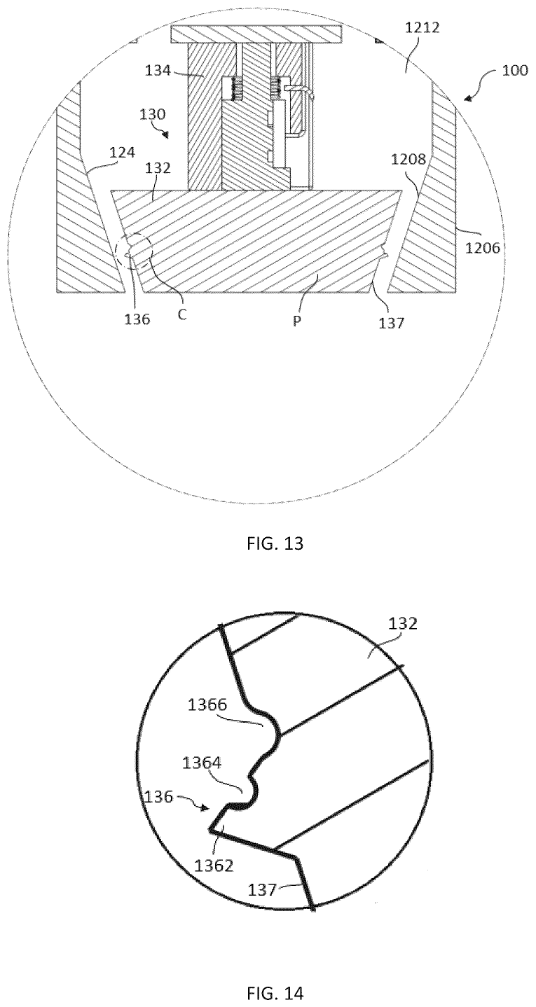

As shown in FIGS. 9 to 14, the sealing unit 130 also includes an elastic seal 136 located between the second opening 122 and the button 132, the clastic seal 136 is arranged around the outer peripheral wall 137 of the button 132 and/or the inner peripheral wall 124 of the second opening 122. In one embodiment, as shown in FIGS. 11 and 12, the elastic seal 136 is arranged around the outer peripheral wall 137 of the button 132; and in another embodiment, as shown in FIGS. 13 and 14, the elastic seal 136 is arranged around the inner peripheral wall 124 of the second opening 122.

The clastic seal 136 is configured to have an interference fit with the outer peripheral wall 137 of the button 132 and the inner peripheral wall 124 of the second opening 122. As shown in FIGS. 9 and 10, when the button 132 is in the first position P′, the elastic seal 136 is compressed and deformed, and tightly adheres to the outer peripheral wall 137 of the button 132 and the inner peripheral wall 124 of the second opening 122. This ensures that when the button 132 is in the first position P′, the sealing unit 130 seals the fragrance assembly 100, and the air inside the carrier 120 does not flow out through the gap 129 between the button 132 and the second opening 122.

As shown in FIGS. 12 and 14, the elastic seal 136 includes a sealing portion 1362 located at an end and a guide groove 1364 adjacent to the sealing portion 1362. The guide groove 1364 is located on one side of the elastic seal 136 facing a movement direction of the elastic seal 136. The movement direction of the elastic seal 136 is opposite to the relative motion direction of the component where the elastic seal 136 is located. When the elastic seal 136 is compressed, the guide groove 1364 guides the sealing portion 1362 to move towards the guide groove 1364.

The sealing unit 130 also includes a receiving groove 1366 arranged on the outer peripheral wall 137 of the button 132 or the inner peripheral wall 124 of the second opening 122. The receiving groove 1366 is tightly pressed against the sealing portion 1362, and the guide groove 1364 and the receiving groove 1366 are located on the same side of the elastic seal 136. In the sealed state, part of the sealing portion 1362 is accommodated in the receiving groove 1366.

In one embodiment, as shown in FIGS. 11 and 12, the elastic seal 136 is arranged around the inner peripheral wall 124 of the second opening 122, the movement direction of the elastic seal 136 is opposite to the relative motion direction of the second opening 122, and the relative motion direction of the second opening 122 is opposite to the first direction, that is, the direction of the elastic seal 136 is the same as the first direction X. The guide groove 1364 is located on the side of the elastic seal facing the first direction X. The sealing portion 1362 extends obliquely towards the outer peripheral wall 137 of the button 132 along a direction opposite to the airflow direction. The receiving groove 1366 is arranged at the inner peripheral wall 124 of the second opening 122, and the guide groove 1364 and the receiving groove 1366 are located on the same side of the elastic seal 136. The guide groove 1364 is located between the receiving groove 1366 and the sealing portion 1362, and the receiving groove 1364 has a size not smaller than that of the sealing portion 1362.

In another embodiment, as shown in FIGS. 13 and 14, the clastic seal 136 is arranged around the outer peripheral wall 137 of the button 132, and the sealing portion 1362 extends obliquely towards the inner peripheral wall 124 of the second opening 122. The movement direction of the elastic seal 136 is opposite to the relative movement direction of the button 132, and the relative movement direction of the button is the same as the first direction X, that is, the direction of the elastic seal 136 is opposite to the first direction X. The guide groove 1364 is located on the side of the elastic seal away from the first direction. Along the direction of the airflow, the sealing portion 1362 extends obliquely towards the inner peripheral wall 124 of the second opening 122. The receiving groove 1366 is arranged at the outer peripheral wall 137 of the button 132, and the guide groove 1364 and the receiving groove 1366 are located on the same side of the elastic seal 136. The guide groove 1364 is located between the receiving groove 1366 and the receiving portion 1362, and the size of the receiving groove 1364 is not smaller than that of the receiving portion 1362.

In the case where the elastic seal 136 adopts this structure, when the fragrance assembly 100 switches from the non-sealed state to the sealed state, the button 132 moves from the second position P to the first position P′, the sealing portion 1362 of the elastic seal 136 moves towards the guide groove 1364, and a part of the sealing portion 1362 is accommodated in the receiving groove 1366. This causes the entire sealing portion 1362, which is arranged in a surrounding manner, to move in the same direction, which can improve the sealing effect and better reduce the possibility of fragrance leakage. And in the sealed state, the sealing portion 1362 can be partially accommodated in the receiving groove 1366, which not only enables better interference fit of the elastic seal 136, but also allows the excess sealing portion to be accommodated in the receiving groove 1366, improving the flexibility of the sealing portion 1362 design, and preventing airflow from flowing through the gap 129 between the second opening 122 and the button 132, further improving the sealing effect.

As shown in FIGS. 2 and 15, a path of airflow passing through the fragrance assembly is indicated by the arrows in FIG. 15. The vehicle fragrance device 100 may further include an airflow channel 1218 for airflow to flow through, and a fan 300 disposed in the airflow channel 1218 for forming airflow between the air inlet 126 and the aroma outlet 128.

As shown in FIG. 3, the vehicle fragrance device 100 may further include a control valve 150 provided in the airflow channel 1218, which is used to open or close the airflow channel 1218. In one embodiment, the control valve 150 can be provided at the air inlet 126 or the aroma outlet 128 of the airflow channel 1218. The control valve 150 is configured to close the airflow channel 1218 when in the sealed state and open the airflow channel 1218 when in the non-sealed state.

In the non-sealed state, the fan 300 is turned on, the control valve 150 is in an open state, and the airflow flowing through the fragrance medium 112 is blown into the vehicle from the aroma outlet 128. In the sealed state, the control valve 150 is in a closed state, and the airflow channel 1218 is closed by the control valve 150, preventing the airflow from flowing out through the airflow channel 1218. This prevents the air inside the accommodating chamber from flowing out through the airflow channel 1218, further reducing the possibility of fragrance leakage.

In one embodiment, the control valve 150 may be a one-way valve. In the non-sealed state, the fan 300 is turned on, the one-way valve is blown open by the airflow, and the airflow channel 1218 is opened, allowing the airflow passing through fragrance medium 112 to flow out through the airflow channel 1218. In the sealed state, the fan 300 is turned off, and the pressure inside the accommodating chamber increases, causing the one-way valve to close and preventing the airflow from flowing out through the airflow channel 1218. In another embodiment, the control valve 150 may be an electronic control valve 150 or a mechanical valve.

Next, with reference to FIGS. 4 to 8, a detailed explanation of how the actuating assembly 200 triggers the fragrance assembly 100 is provided.

The vehicle fragrance device 10 may include multiple fragrance assemblies 100. Different fragrance assemblies 100 are equipped with fragrance medium 112 with different scent flavors. In one embodiment, the actuating assembly 200 can activate one of the fragrance assemblies 100 to be in the non-sealed state and the remaining fragrance assemblies 100 are in the sealed state, which allows the user to select a single type of fragrance. In another embodiment, the actuating assembly 200 can activate two or more fragrance assemblies 100 to be in the non-sealed state, while the remaining fragrance assemblies 100 are in the sealed state, which allows the user to choose different types of mixed fragrances as needed. Technicians in this field design different triggered positions to activate one or more fragrance assemblies according to actual needs, and these variations should also be within the scope of protection of the present disclosure without departing from the spirit of protection of the present disclosure.

As mentioned above, the actuating assembly 200 may also include an actuation rod 220, on which the actuator 210 is mounted. The actuating assembly 200 may also include an actuating motor 230, a gear 240, and a rack 250 that cooperates with the gear 240. The actuating motor 230 can drive the gear 240 to rotate, and then drive the rack 250 to move through the gear 240, thereby driving the actuator 210 to move. Through the cooperation of the actuator 210 and the scaling unit 130, the fragrance assembly 100 can be turned on or off to release the specified fragrance.

Each actuator 210 described above has corresponding triggered and non-triggered positions, and the actuator 210 can move between the triggered position and the non-triggered position. When in the triggered position, the actuator 210 triggers the corresponding sealing unit 130, and when in the non-triggered position, the actuator 210 disengages from the sealing unit 130. When a specified fragrance is required, the actuator 210 is moved to the triggered position of the fragrance assembly 100 corresponding to the specified fragrance under the action of the actuating motor 230 to trigger the sealing unit 130, the fragrance assembly 100 corresponding to the specified fragrance is triggered to switch to the non-sealed state, while the remaining fragrance assemblies 100 are in the sealed state.

As shown in FIGS. 3 and 4, in one embodiment, multiple actuator 210 are linearly and uniformly arranged on the actuating rod 220, and the number of the actuators 210 may be consistent with the number of the fragrance assemblies 100. The actuating rod 220 is configured to perform reciprocating motion along the extension direction of the actuating rod 220 and drive the actuator 210 to reciprocate. The spacing between the actuators 210 is not equal to the spacing between the fragrance assemblies 100. This can enable when one actuator 210 of the actuating assembly 200 is in the triggered position, the other actuators 210 are in the non-triggered position, so that only one actuator 210 can trigger the corresponding fragrance assembly 100 to switch between the sealed and non-sealed states, to release a single fragrance. It can be understood that the spacing between the actuators 210 can be adjusted as needed, so that two or more brakes can be located at the triggered position at a time, and two or more fragrance assemblies 100 can be triggered to switch between the sealed and unsealed states, and thus the vehicle fragrance device can release mixed fragrances when switching fragrances. In another embodiment, the spacing between the actuators 210 is smaller than the spacing between fragrance assemblies 100. This not only enables the actuating assembly 200 to trigger only one fragrance assembly 100 at a time, but also reduces the spacing between the actuators 210, making the actuating assembly 200 more compact and enhancing space utilization.

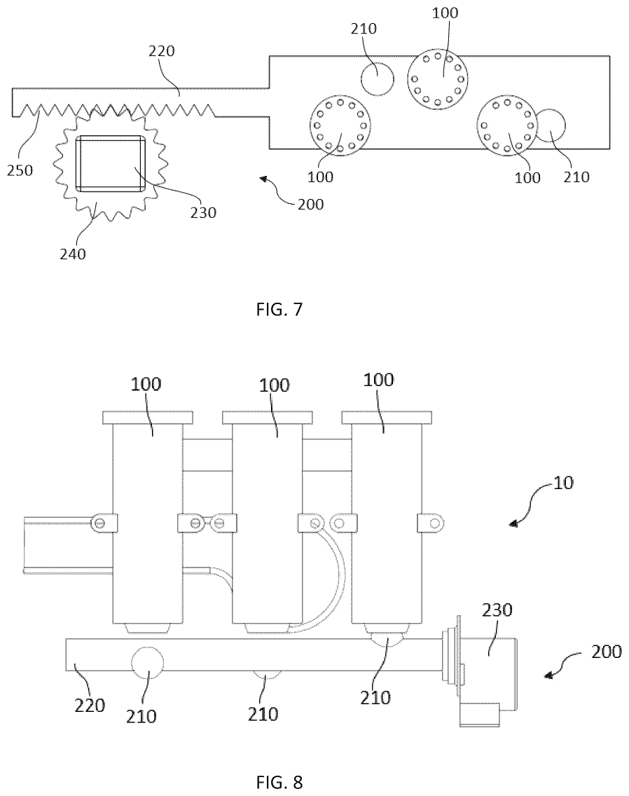

In yet another embodiment, as shown in FIG. 7, multiple actuators are nonlinearly arranged on the actuating rod. This can make the actuating assembly design more compact. Multiple fragrance assemblies are arranged nonlinearly, and the actuator is constructed such that when one of the fragrance assemblies is in the non-scaled state, the remaining fragrance assemblies are in the sealed state. Technicians in this field can arrange the actuators and fragrance assemblies as needed. This can make the design of fragrance assemblies and actuating assemblies more flexible and compact, further making the vehicle fragrance device more compact and enhancing space utilization.

In a specific exemplary embodiment, the number of the actuators 210 and the number of the fragrance assemblies 100 is three (3) each. However, other numbers of actuators 210 and fragrance assemblies may be included in the vehicle fragrance device 10.

After triggering the sealing unit 130, the actuator 210 disengages from the sealing unit 130 and the fragrance assembly 100 remains in the triggered state 210. It can be understood that only one actuator 210 can be provided, while one or more fragrance assemblies 100 can be provided. When the user needs to specify a single fragrance, the actuator 210 is moved to the corresponding triggered position to trigger the corresponding fragrance assembly 100 to switch to the non-scaled state. The brake then disengages from the scaling unit 130 and can be moved by the motor to trigger other fragrance assemblies 100, causing them to switch into the sealed state.

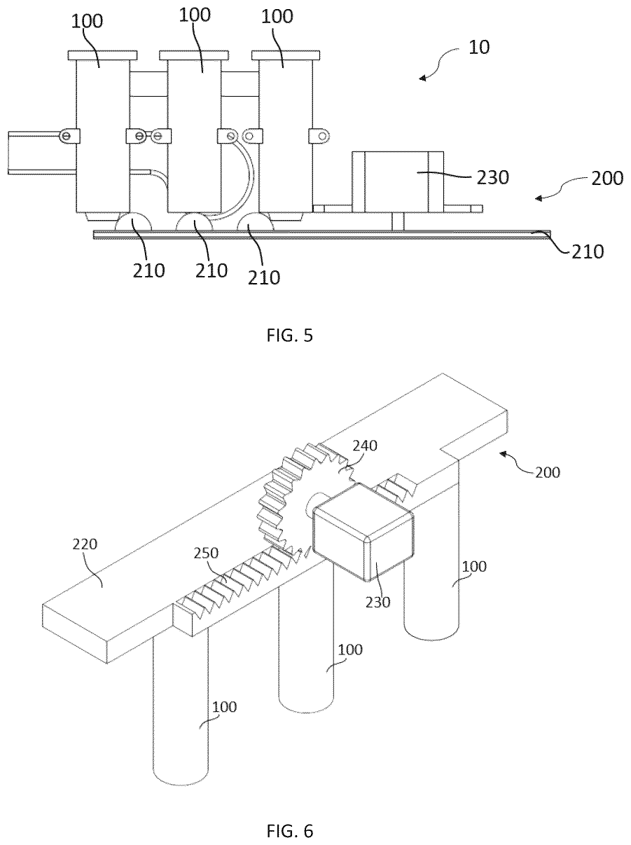

In another embodiment, as shown in FIG. 6, the actuating rod 220 is driven to rotate along its central axis under the drive of the motor, and the actuating rod 220 drives the actuator 210 to rotate along the central axis of the actuating rod 220, causing the actuator 210 to move between the triggered position and the non-triggered position. The actuators 210 have the same number as the fragrance assemblies 100, and along the extension direction of the actuating rod 220, the actuators 210 are arranged at the position corresponding to the fragrance assembly 100 on the actuating rod 220. The actuators 210 are arranged along a circumferential direction of the actuating rod 220. When there are multiple actuators 210, projections of the actuators 210 on the plane perpendicular to the central axis of the actuating rod 220 are at least partially offset. Thus, only one actuator 210 is located at the triggered position at a time, and only one fragrance assembly 100 can switch between the sealed and unsealed states to release a single fragrance. It can be understood that the positions of the actuators 210 along the circumferential direction of the actuating rod 220 can be adjusted as needed, so that the projections of two or more actuators 210 on a plane perpendicular to the central axis of the actuating rod 220 coincide. This allows for the simultaneous triggering of two or more fragrance assemblies 100 to switch between the sealed and unsealed states. When switching fragrances, the fragrance device can release mixed fragrances.

As shown in FIG. 1, another aspect of the present disclosure also provides a vehicle 400 equipped with the above-mentioned vehicle fragrance device 10. The vehicle fragrance device 10 can be installed at different positions inside the vehicle 400. In one embodiment, the fragrance device can be installed on a central armrest or a rear armrest for user control, to turn on or off the fragrance device. In another embodiment, the fragrance device can also be installed on a dashboard. In yet another embodiment, the fragrance device can also be installed on a roof console to enhance the user's perception of fragrance and further enhance the user experience.

The present disclosure summarizes aspects of the embodiments and should not be used to limit the claims. Other implementations are contemplated in accordance with the techniques described herein, as will be apparent upon examination of the following drawings and detailed description, and such implementations are intended to be within the scope of this application.

The present disclosure provides a vehicle fragrance device and a vehicle. The vehicle fragrance device includes a fragrance assembly having a fragrance unit with a fragrance medium, a carrier for carrying the fragrance unit, and a sealing unit; and an actuating assembly comprising a movable actuator, wherein the actuator triggers the sealing unit to switch the fragrance assembly between a sealed state and a non-sealed state, and after triggering the sealing unit, the actuator disengages from the sealing unit and the fragrance assembly remains in the triggered state.

According to an embodiment of the present disclosure, the sealing unit further comprises a button and a push lock connected to the button, the button has a first position corresponding to the sealed state and a second position corresponding to the non-sealed state, the button is configured to trigger the actuator to switch the button between the first position and the second position, and the push lock locks the button in the triggered position.

According to an embodiment of the present disclosure, the carrier comprises a bottom wall and a peripheral wall around the bottom wall, the carrier further comprises a peripheral wall extension end, the peripheral wall extension end extends away from the fragrance unit from the bottom wall and has an inner inclined surface adapted to the button and facing the button.

According to an embodiment of the present disclosure, the carrier has a first accommodating chamber for accommodating the fragrance unit and a second accommodating chamber located at an end of the carrier and being in communication with the first accommodating chamber, and the sealing unit is located in the second accommodating chamber.

According to an embodiment of the present disclosure, the carrier has a first opening facing the actuator, and the sealing unit seals the first opening in the sealed state.

According to an embodiment of the present disclosure, the sealing unit further comprises a button and an elastic seal located between the opening and the button, and the elastic scal is arranged around an outer peripheral wall of the button and/or an inner peripheral wall of the opening.

According to an embodiment of the present disclosure, the elastic seal comprises a scaling portion located at an end and a guide groove adjacent to the sealing portion, and the guide groove is located on one side of the elastic seal facing a movement direction of the elastic seal.

According to an embodiment of the present disclosure, the sealing unit further comprises a receiving groove arranged on the outer peripheral wall of the button or the inner peripheral wall of the opening, the sealing portion rests against the receiving groove, the guide groove and the receiving groove are located on the same side of the clastic seal, and the scaling portion is partially accommodated in the receiving groove in the sealed state.

According to an embodiment of the present disclosure, the elastic seal comprises a scaling portion located at an end and a guide groove adjacent to the sealing portion, and the guide groove guides the sealing portion to move towards the guide groove when the elastic seal is compressed.

According to an embodiment of the present disclosure, the carrier has an air inlet and an aroma outlet, and the sealing unit is used to seal the air inlet and/or aroma outlet.

According to an embodiment of the present disclosure, the fragrance assembly further comprises an airflow channel for airflow to pass through and a control valve provided in the airflow channel.

According to an embodiment of the present disclosure, the actuating assembly further comprises an actuating rod, on which the actuator is mounted, and the actuator is configured to perform reciprocating motion along an extension direction of the actuating rod to trigger or disengage from the sealing unit.

According to an embodiment of the present disclosure, the actuating assembly comprises multiple linearly and uniformly arranged actuators, and the spacing between the actuators is not equal to the spacing between the fragrance assemblies.

According to an embodiment of the present disclosure, the spacing between the actuators is smaller than the spacing between the fragrance assemblies.

According to an embodiment of the present disclosure, multiple actuators are nonlinearly arranged on the actuating rod.

According to an embodiment of the present disclosure, multiple fragrance assemblies are arranged nonlinearly, and the actuator is configured such that when one of the fragrance assemblies is in the non-sealed state, the remaining fragrance assemblies are in the sealed state.

According to an embodiment of the present disclosure, the actuating assembly further comprises an actuating rod, on which the actuator is arranged, and the actuator is configured to perform rotational motion along a central axis of the actuating rod to trigger or disengage from the scaling unit.

According to an embodiment of the present disclosure, multiple actuators adapted to the positions of the fragrance assemblies are provided, and projections of the multiple actuators on a plane perpendicular to the central axis of the actuating rod are at least partially offset.

According to an embodiment of the present disclosure, the fragrance assembly further comprises a cover, the cover comprises a main body portion and a connecting portion that is connected to the carrier on one side in a sealed manner and to the fragrance unit on the other side.

According to an embodiment of the present disclosure, the fragrance unit comprises a fragrance box, the fragrance medium is located inside the fragrance box, and a first fragrance retention gap is formed between the fragrance box and the carrier.

Another aspect of the present disclosure also provides a vehicle comprising a vehicle fragrance device located inside the vehicle, the fragrance device comprises: a housing that at least partially accommodates a fragrance assembly, the fragrance assembly comprising a fragrance unit with a fragrance medium, a carrier for carrying the fragrance unit, and a scaling unit; and an actuating assembly comprising a movable actuator, wherein the actuator triggers the sealing unit to switch the fragrance assembly between a sealed state and a non-sealed state, and after triggering the sealing unit, the actuator disengages from the sealing unit and the fragrance assembly remains in the triggered state.

Additional features of the present disclosure can be derived from the claims, drawings, and description of the drawings. The features and feature combinations previously named in the specification, as well as the features and feature combinations named in the following description of the drawings and/or separately shown in the following drawings, may not only be used in the corresponding indicated combinations, but also in other combinations or separately, without departing from the scope of the present disclosure. Therefore, the embodiments of the present disclosure will also be considered to include and disclose content that is not explicitly shown or illustrated in the accompanying drawings, but is derived from and can be generated from the separated feature combinations in the illustrated embodiments.

The above-mentioned embodiments are possible examples of implementations of the present disclosure and are given only for the purpose of enabling those skilled in the art to clearly understand the principles of the disclosure. It should be understood by those skilled in the art that the above discussion to any embodiment is only illustrative, and is not intended to imply that the disclosed scope of the embodiments of the present disclosure is limited to these examples. Under the overall concept of the disclosure, the technical features in the above embodiments or different embodiments can be combined with each other to produce many other their variants in different aspects of embodiments of the disclosure that is not provided in detailed description for the sake of brevity. Therefore, any omission, modification, equivalent replacement, improvement, etc. made within the spirit and principle of the embodiment of the disclosure shall be included in the scope of protection claimed.

It is to be understood that variations and modifications can be made on the aforementioned structure without departing from the concepts of the present disclosure, and further it is to be understood that such concepts are intended to be covered by the following claims unless these claims by their language expressly state otherwise.

What is claimed is:

1. A vehicle fragrance device, comprising:
a fragrance assembly comprising a fragrance unit with a fragrance medium, a carrier for carrying the fragrance unit, and a sealing unit; and
an actuating assembly comprising a movable actuator, wherein movable the actuator triggers the sealing unit to switch the fragrance assembly between a sealed state and a non-sealed state, and after triggering the sealing unit, the movable actuator disengages from the sealing unit and the fragrance assembly remains in the triggered state.

2. The vehicle fragrance device as claimed in claim 1, wherein the sealing unit further comprises a button and a push lock connected to the button, the button has a first position corresponding to the sealed state and a second position corresponding to the non-sealed state, and wherein the button is configured to trigger the actuator to switch the button between the first position and the second position, and the push lock locks the button in the triggered position.

3. The vehicle fragrance device as claimed in claim 2, wherein the carrier comprises a bottom wall and a peripheral wall around the bottom wall, and wherein the carrier further comprises a peripheral wall extension end, the peripheral wall extension end extends away from the fragrance unit from the bottom wall and has an inner inclined surface adapted to the button and facing the button.

4. The vehicle fragrance device as claimed in claim 1, wherein the carrier has a first accommodating chamber for accommodating the fragrance unit and a second accommodating chamber located at an end of the carrier and being in communication with the first accommodating chamber, and the sealing unit is located in the second accommodating chamber.

5. The vehicle fragrance device as claimed in claim 1, wherein the carrier has a first opening facing the movable actuator, and the sealing unit seals the first opening in the sealed state.

6. The vehicle fragrance device as claimed in claim 5, wherein the sealing unit further comprises a button and an elastic seal located between the opening and the button, and the elastic seal is arranged around at least one of an outer peripheral wall of the button and an inner peripheral wall of the opening.

7. The vehicle fragrance device as claimed in claim 6, wherein the elastic seal comprises a sealing portion located at an end and a guide groove adjacent to the sealing portion, and the guide groove is located on one side of the elastic seal facing a movement direction of the elastic seal.

8. The vehicle fragrance device as claimed in claim 7, wherein the sealing unit further comprises a receiving groove arranged on the outer peripheral wall of the button or the inner peripheral wall of the opening, and wherein the sealing portion rests against the receiving groove, the guide groove and the receiving groove are located on the same side of the elastic seal, and the sealing portion is partially accommodated in the receiving groove in the sealed state.

9. The vehicle fragrance device as claimed in claim 1, wherein the carrier has an air inlet and an aroma outlet, and the sealing unit is used to seal at least one of the air inlet and the aroma outlet.

10. The vehicle fragrance device as claimed in claim 1, further comprising an airflow channel for airflow to pass through and a control valve provided in the airflow channel.

11. The vehicle fragrance device as claimed in claim 1, wherein the actuating assembly further comprises an actuating rod, on which the movable actuator is arranged, and the movable actuator is configured to perform reciprocating motion along an extension direction of the actuating rod to trigger or disengage from the sealing unit.

12. The vehicle fragrance device as claimed in claim 11, wherein the actuating assembly comprises multiple linearly and uniformly arranged actuators, and a spacing between the actuators is not equal to a spacing between the fragrance assemblies.

13. The vehicle fragrance device as claimed in claim 12, wherein the spacing between the actuators is smaller than the spacing between the fragrance assemblies.

14. The vehicle fragrance device as claimed in claim 11, wherein multiple actuators are nonlinearly arranged on the actuating rod.

15. The vehicle fragrance assembly as claimed in claim 11, wherein multiple fragrance assemblies are arranged nonlinearly, and the actuator is configured such that when one of the fragrance assemblies is in the non-sealed state, the remaining fragrance assemblies are in the sealed state.

16. The vehicle fragrance device as claimed in claim 1, wherein the actuating assembly further comprises an actuating rod, on which the actuator is mounted, and the movable actuator is configured to perform rotational motion along a central axis of the actuating rod to trigger or disengage from the sealing unit.

17. The vehicle fragrance device as claimed in claim 16, wherein multiple actuators adapted to the positions of the fragrance assemblies are provided, and projections of the multiple actuators on a plane perpendicular to the central axis of the actuating rod are at least partially offset.

18. The vehicle fragrance device as claimed in claim 1, wherein the fragrance assembly further comprises a cover comprising a main body portion and a connecting portion that is connected to the carrier on one side in a sealed manner and to the fragrance unit on another side.

19. The vehicle fragrance device as claimed in claim 18, wherein the fragrance unit comprises a fragrance box, wherein the fragrance medium is located inside the fragrance box, and a first fragrance retention gap is formed between the fragrance box and the carrier.

20. A vehicle comprising a vehicle fragrance device located inside the vehicle, the fragrance device comprising:
a housing that at least partially accommodates a fragrance assembly;
the fragrance assembly comprising a fragrance unit with a fragrance medium, a carrier for carrying the fragrance unit, and a sealing unit; and
an actuating assembly comprising a movable actuator, wherein the movable actuator triggers the sealing unit to switch the fragrance assembly between a sealed state and a non-sealed state, and after triggering the sealing unit, the movable actuator disengages from the sealing unit and the fragrance assembly remains in the triggered state.
